(19) United States
(12) Patent Application Publication
(10) Pub. No.: US 2025/0049394 A1
(43) Pub. Date: Feb. 13, 2025

(54) CUSTOMIZED INTERWOVEN WEARABLE WITH SENSOR POSITIONING

(71) Applicant: Aspen Medical Products, LLC, Irvine, CA (US)

(72) Inventors: Harry Duane Romo, Pahrump, NV (US); Robert Matthew Damstra, Lake Forest, CA (US); Albert V. Romo, Lakewood, CA (US); Joel Perez, Long Beach, CA (US)

(21) Appl. No.: 18/448,923

(22) Filed: Aug. 12, 2023

(51) Int. Cl. A61B 5/00; A61B 5/01; A61B 5/11

(52) U.S. Cl. CPC A61B 5/6812; A61B 5/01; A61B 5/1117; A61B 5/445; A61B 2560/0462; A61B 2562/0219; A61B 2562/029

(57) ABSTRACT

One embodiment relates to an orthopedic precast featuring sensors to detect certain a condition of the patient and usage of an orthosis produced from the orthopedic precast. The orthopedic precast features (i) a first portion including a first plurality of interwoven strands of a first thermoplastic material, (ii) a second portion coupled to the first plurality of interwoven strands of the first thermoplastic material, and (iii) one or more sensors. The first thermoplastic material has a first melting temperature. Responsive to applying heat of at least the first melting temperature to the first portion to cause partial melting of interwoven strands of the first thermoplastic material and subsequent cooling thereafter, the interwoven strands of the first thermoplastic material transition from a flexible state to a rigid state to create one or more recessed area for receipt of the one or more sensors.

### FIELD

One embodiment of the disclosure relates to orthopedic braces (orthoses) and wearables with hardening thermoplastic material configured for sensor deployment and retention.

### BACKGROUND

The following description includes information that may be useful in understanding the present invention. It is not an admission that any of the information provided herein is prior art or relevant to the presently claimed invention, or that any publication specifically or implicitly referenced is prior art.

Orthopedic braces (orthoses) usually need to be adjusted or customized in some manner to conform to the body part(s) being braced, and then properly positioned. A typical orthosis commonly has at least two portions, namely a rigid portion operating as a structural support and a flexible portion in contact with the body. However, these conventional orthoses are static in design and cannot be easily customized for a patient. Moreover, these orthoses do not allow for needed relief areas particular to account for anatomical differences between patients, and thus, orthotists sometimes need to conduct time-consuming operations in disassembling, adding to or removing portions from the orthosis to create much needed relief areas.

Custom orthoses have been made from a combination of resilient and flexible materials. For example, U.S. Pat. No. 9,572,703 to Matthews describes an orthosis sock that utilizes a resilient material for restricting movement of a patient's foot. Because the orthosis is a sock, it is relatively easier to wear than a typical ankle-foot-orthosis (AFO). However, resiliency of the material can be insufficient to provide adequate support and the stitching of the resilient material with Lycra® elastomeric material is both labor intensive and susceptible to uncomfortable areas at the stitching points. Also, these customized orthoses do not deploy sensor technology to monitor usage of an orthosis as well as to monitor movement and/or health conditions of the patient.

Currently, custom orthoses are formed by creating a negative mold of a patient's body part, using the negative mold to create a positive mold, wrapping preheated flexible and hardenable materials about different portions of the positive mold, and then applying a vacuum to the material-wrapped positive mold and allowing time for the materials to cool to the shape of the positive mold. Once cooled, the materials must be carefully cut off the mold then all cut edges must be ground/smoothed to the final shape. Production of such custom orthosis is extremely labor intensive, time consuming, expensive and requires considerable skill that is in short supply in the marketplace. Moreover, there is no fabrication within materials that are used to form the orthosis to account for sensors deployed therein.

Thus, there is still a need for systems and methods for efficiently producing custom orthoses with sensor technology positioned in a secure member within the orthosis.

### BRIEF DESCRIPTION OF THE DRAWINGS

FIG. 1A is a perspective view of an exemplary embodiment of a precast for a cervical orthosis interweaved to retain sensors to monitor usage and health conditions.

FIG. 1B is a perspective view of the cervical orthosis created from the precast of FIG. 1A.

FIG. 2A is a schematic of a first exemplary embodiment of a portion of the orthopedic precast of FIG. 1A with strands of thermoplastic material formed as a layer of the cervical orthosis.

FIG. 2B is a schematic of the orthopedic precast portion of FIG. 2A in which the strands of thermoplastic material are heated to form a hardened structure of the cervical orthosis with interior-facing sensor deployment.

FIG. 2C is a schematic of the orthopedic precast portion of FIG. 2A in which the strands of thermoplastic material are heated to form a hardened structure of the cervical orthosis with exterior-facing sensor deployment.

FIG. 2D is a schematic of an orthosis generated from the orthopedic precast portion of FIG. 2C.

FIG. 3A is an exemplary embodiment of an orthopedic precast with deployed sensors operating as a lumbar sacral orthosis (LSO) or part of a thoracic lumbar sacral orthosis (TLSO).

FIG. 3B is an exemplary embodiment of the orthosis produced from the orthopedic precast of FIG. 3A.

FIG. 4 is an exemplary embodiment of an orthopedic precast of a leg-ankle-foot orthosis with sensor deployment.

FIG. 5A is a second exemplary embodiment of a portion of an orthopedic precast with interwoven strands of thermoplastic material with sensors organized in a grid configuration.

FIG. 5B is an exemplary embodiment of an orthosis resulting from the orthopedic precast of FIG. 5A.

FIG. 6 is an exemplary embodiment of a system featuring a computing device adapted for communications with sensors associated with one or more orthoses and analytics of data captured by the sensors.

### DETAILED DESCRIPTION

The following discussion provides many example embodiments of the inventive subject matter. Although each embodiment represents a single combination of inventive elements, the inventive subject matter is considered to include all possible combinations of the disclosed elements. Thus, if one embodiment comprises elements A, B, and C, and a second embodiment comprises elements B and D, then the inventive subject matter is also considered to include any combination of A, B, C, and/or D, even if not explicitly disclosed.

In some embodiments, the numbers expressing quantities of ingredients, properties such as concentration, reaction conditions, and so forth, used to describe and claim certain embodiments of the invention are to be understood as being modified while still retaining the spirit and scope of the disclosed invention. Accordingly, in some embodiments, the numerical parameters set forth in the written description and attached claims are illustrative approximations that can vary depending upon the desired properties sought to be obtained by a particular embodiment.

Unless the context dictates the contrary, all ranges set forth herein should be interpreted as being inclusive of their endpoints, and open-ended ranges should be interpreted to include only commercially practical values. Similarly, all lists of values should be considered as inclusive of intermediate values unless the context indicates the contrary.

While each inventive aspect of the disclosure may be subject to various modifications, equivalents, and alternative forms, specific embodiments thereof have been shown by way of example in the drawings and will now be described in detail. It should be understood that each inventive aspect is not limited to the particular embodiments disclosed, but in contrast, the intention is to cover modifications, equivalents, and alternative forms of the inventive aspects within the specific embodiments as each inventive aspect may be implemented into any of these different embodiments.

### I. General Summary

The inventive subject matter pertains to a wearable formed by interwoven materials including strands of thermoplastic materials that, after undergoing a phase change, create pathways (e.g., recessed areas, channels, etc.) for retaining electronic components (e.g., sensor, wireless transceiver, wireless transmitter, etc.) therein. In some embodiments, the wearable constitutes an orthopedic precast featuring strands of thermoplastic material interwoven into regions of the precast. The strands of thermoplastic material may be interwoven in accordance with a particular arrangement so that, upon applying heat at or exceeding a prescribed temperature, the thermoplastic material partially melts and diffuses to create recessed areas within the orthopedic precast. After cooling, these recessed areas form sensor housings within rigid structures of the resultant orthosis.

According to one embodiment of the disclosure, a customized orthosis is created to include (i) one or more rigid structures with one or more sensor housings and (ii) one or more elastic structures that are devoid of the thermoplastic material or feature thermoplastic material having a greater melting temperature than the heat applied to create a rigid (hardened) structure. The elastic material may be arranged to be in contact with the patient. Examples of an orthopedic precast, namely the original state of a collection of materials used to create a custom orthosis, may include, but are not limited or restricted the following: a cervical orthosis (e.g., cervical collar), an ankle-foot-orthosis (AFO), a torso orthosis, a leg-ankle-foot orthosis, a wrist orthosis, a shoulder orthosis, or the like.

According to one embodiment of the disclosure, the orthopedic precast may feature (i) a first portion comprising one or more interwoven strands of at least a first thermoplastic material that is attached to (ii) a second portion formed with interwoven strands of non-thermoplastic material, lesser strands of thermoplastic material, or strands of a different thermoplastic material. The first interwoven portion and the second interwoven portion may be attached as part of a single layer precast construction, where portions of the precast utilize different thermoplastic materials, different interweaving techniques, and/or different strand thicknesses to provide a structural framework for the orthosis along with rigid recessed areas for sensors. Other portions of the orthopedic precast may feature material with flexible and/or elastic construction. The first interwoven portion and/or the second interwoven portion may be layered to provide additional rigidity as needed.

When applying a prescribed amount of heat to specific regions of the orthopedic precast, one or more of these interwoven portions (hereinafter, “interwoven portion(s)”) may experience a phase transition, becoming more rigid than its original construction. For example, the first thermoplastic material may undergo a phase change by partially melting in response to an application of heat at or above a melting temperature for the first thermoplastic material. This application of heat to specific regions inclusive of the first thermoplastic material may be accomplished through direct heating (e.g., heated air flow, heated environment, etc.), an application of steam (i.e., combination of heat and water vapor), or thermal conduction with heat being directly applied or radiated by a powered heating element within the orthopedic precast.

After experiencing a phase transition and subsequent cooling, the first interwoven portion transitions into a “shell” portion, which provides rigidity within a region of the resultant orthosis corresponding to the first interwoven region. The shell portion may include recessed areas produced from the pattern of interwoven strands of the first thermoplastic material to partially house and protect sensors deployed as part of the resultant orthosis. The sensors may constitute (i) an accelerometer to detect a change in gravitational acceleration, making it possible to measure tilt, vibration (e.g., leg weakness, neck weakness, etc.), or a fall, (ii) moisture sensor to detect a lack of dryness on a wound or surgical incision area, (iii) motion sensor to detect usage of the orthosis, or any combination thereof.

Herein, different interwoven portions may experience different amount of phase change, depending on its material composition and structure. The different degrees of phase change may provide regions of the orthosis with different levels of rigidity (e.g., rigid, flexible, etc.) where deployment in one type may improve operability of a sensor. As an illustrative example, for the situation where a second interwoven portion includes the same thermoplastic material as a first interwoven portion, but in lesser quantity (e.g., lesser strand numbers, thinner thermoplastic strands, lesser volume of thermoplastic strands due to interweaving/stitching pattern, etc.), after activation (e.g., heating to cause at least partial melting of the thermoplastic material), the first interwoven portion becomes more rigid than the second interwoven portion. For sensors that require precise deployments (e.g., tilt sensors whose accuracy may be influenced by sway caused by larger than needed tolerances in the sensor housing, etc.), the strands of thermoplastic material interwoven in the first interwoven portion may be arranged to receive the tilt sensors while the strands of thermoplastic material interwoven in the second interwoven portion may be arranged to receive moisture sensor as the more flexible (less rigid) regions are likely closer to the skin of the patient.

Herein, according to one embodiment of the disclosure, melting temperatures of thermoplastic materials used in a precast may range from 500 Celsius (C) to 150° C. The differences between melting temperatures of different thermoplastic materials may vary from ten degrees Celsius (10° C.) or more, such as from 10° C.-50° C., or even greater.

In some embodiments, the thermoplastic material composition can comprise at least 30 wt % of a precast. For example, the thermoplastic portion can comprise between 5 wt % and 90 wt % of the orthopedic precast, more preferably between 50 wt % and 90 wt % of the orthopedic precast, still more preferably between 80 wt % and 90 wt % of the orthopedic precast.

### II. Terminology

As used herein, the terms “interweaving,” “interweaved,” “interwoven,” or any other tenses thereof are generally defined as any arrangement of material substantially comprising one or more strands of material (e.g., thread, yarn, etc.) to produce a custom wearable (e.g., orthopedic precast to be converted to an orthosis, clothing, etc.). The arrangement of material may include an attachment of one or more types of materials together as a single layer of continuous material or multiple (two or more) layers of material, where the attachment may occur through knitting, stitching (e.g., “V” shaped stiches), weaving (interlacing strands of material), crocheting (knot-like stitches), macrame (knot-like stiches in geometrical patterns), and/or spray-on application of thermoplastic material to alter the composition of the material such as from a non-thermoplastic material to a thermoplastic material.

Furthermore, this interweaving of material may be performed in accordance with a two-dimensional (2D) interweaving process or a three-dimensional (3D) interweaving process. The 2D interweaving process allows for the creation of a low-profile orthoses where the shell portion and flexible portion are interwoven to form a single layer of material. The 3D interweaving process may be utilized to create a layered interwoven orthosis, such as added padding and/or additional layers of the same or different types of thermoplastic material may be added to the shell portion to increase its rigidity. Also, the increased layering provides increased density for larger-sized orthoses or in certain areas of the orthoses that need increased density (e.g., hip area, etc.).

As used herein, the terms “rigid” or “rigidity” with respect to a wearable or a portion of the wearable, such as an orthopedic precast or clothing, means that the rigid wearable or rigid portion of the wearable will resist bending or deformation. According to this definition, different lengths of a given structure and composition can be rigid at a shorter length, and flexible at a longer length. As an illustrative example, in some cases, a “rigid” object or “rigid” portion(s) of the object may represent that the object or the portion of the object may be deformed, in some cases permanently (i.e., broken), if bent or twisted by a predetermined angle. For some levels of rigidity, the predetermined angle may be at least 300 end to end. For other levels of rigidity, where the object or the portion of the object may be considered to be semi-rigid, a bending or twisting over 900 (and perhaps recurrent bending or twisting) would be needed to cause deformation. As used herein, the terms “bent” or “bending” may be construed to applying an angular deformation, which may include twisting.

As used herein, the term “flexible” with respect to a wearable or a portion of the wearable may signify that the wearable or the wearable portion will not be permanently deformed by bending. For example, an interwoven portion of a precast may be flexible in nature, even after a heating and cooling process. As used herein, the term “permanently deformed” means that deformation remains unless the deformation is actively repaired. According to this definition, the wearable or a portion of the wearable could be rigid in one direction, and flexible in another direction. Unless otherwise specified in such cases, the wearable or portion of the wearable is deemed to be rigid.

As used herein, the term “elastic” with respect to a wearable or portion of the wearable means that if the elastic portion is stretched or compressed lengthwise by at least 10%, it will return to its resting length, without the need for application of an external force, and without permanent deformation.

As used herein, the term “shell” means a structure configured to impart rigidity that restrains movement of a part of a patient's body, wherein the structure is either (i) rigid after an application of a prescribed heating and cooling phase or (ii) will become rigid upon completion of that heating and cooling phase. In particular, an interwoven portion of a precast existing in a non-rigid state may constitute a shell portion by having characteristics that allow this interwoven portion to transition from its non-rigid form to a rigid form in response to an application of heat (heated air, steam, etc.) that is equal to or exceeds a prescribed melting temperature and subsequent cooling. In some embodiments, the shell may include a structure having a cavity, hollow, or lumen.

As used herein the term “patient” includes both humans and animals, independently of whether the patient is under the care of a medical or veterinary professional.

As used herein, the term “strand” is generally defined as an elongated length of one or more natural, artificial, or combined natural and artificial substances. According to one embodiment of the disclosure, each strand may be a fiber of a prescribed width (e.g., no more than 3 millimeters (mm) thick over a length of at least one centimeter (cm), although other sizing is contemplated). Examples of a “strand” may include thread, yarn, string, cord, or any flexible material that may be organized into a structure (e.g., stitched, weaved, etc.) to produce an object such as an orthopedic precast (for transformation into an orthosis), or the like. Where the strand or strands denote thermoplastic material, these strands may be elongated fibers of the thermoplastic material, or alternatively, the strand may be another substance at least partially coated with thermoplastic material or impregnated with thermoplastic material.

The term “computing device” should be generally construed as electronics with the data processing capability and/or a capability of connecting to any type of network, such as a public network (e.g., Internet), a private network (e.g., a wireless data telecommunication network, a local area network “LAN,” etc.), or a combination of networks. Examples of a computing device may include, but are not limited or restricted to, the following: an endpoint device (e.g., a laptop, a smartphone, a tablet, a desktop computer, a netbook, a medical device, or any general-purpose or special-purpose, user-controlled electronic device configured to support virtualization), a server a mainframe, or a router.

The computing device includes hardware and/or software modules that are configured to perform certain operations, including analytics of data provided from monitored sources. These software modules may be stored in any type of a suitable non-transitory storage medium. Examples of non-transitory storage medium may include, but are not limited or restricted to, a programmable circuit; semiconductor memory; non-persistent storage such as volatile memory (e.g., any type of random-access memory “RAM”); or persistent storage such as non-volatile memory (e.g., read-only memory “ROM,” power-backed RAM, flash memory, phase-change memory, etc.), a solid-state drive, hard disk drive, an optical disc drive, or a portable memory device.

In some embodiments, the orthopedic precast may be transformed into an orthosis having a construction that includes both shell and flexible portions. In some embodiments, the orthosis may be configured with a tubular construction that includes shell portions of different levels of rigidity.

In some embodiments, the shell portion orients lengthwise along the tube. For example, the shell and flexible portions of a precast could correspond to the anterior and posterior portions of a lower leg respectively, and these portions can be directly coupled to each other. For such a precast, the shell portion would be considered to be oriented lengthwise along the tube. Alternatively, a shell portion can orient crosswise with respect to a tube. For example, a precast capable of accommodating a torso portion of a patient could have a shell portion that extends across the front of a patient, and flexible portions that also extend across the front of the patient, connected superiorly and inferiorly to the shell portion. For such an orthosis, the shell portion would be considered to be oriented crosswise along the tube. As used herein, the term “crosswise” includes various degrees of diagonality.

As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

Finally, the terms “or” and “and/or” as used herein are to be interpreted as inclusive or meaning any one or any combination. As an example, “A, B or C” or “A, B and/or C” mean “any of the following: A; B; C; A and B; A and C; B and C; A, B and C.” An exception to this definition will occur only when a combination of elements, functions, steps, or acts are in some way inherently mutually exclusive.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Various objects, features, aspects, and advantages of the inventive subject matter will become more apparent from the following detailed description of preferred embodiments, along with the accompanying drawing figures in which like numerals represent like components.

### III. Illustrative Orthopedic Precasts

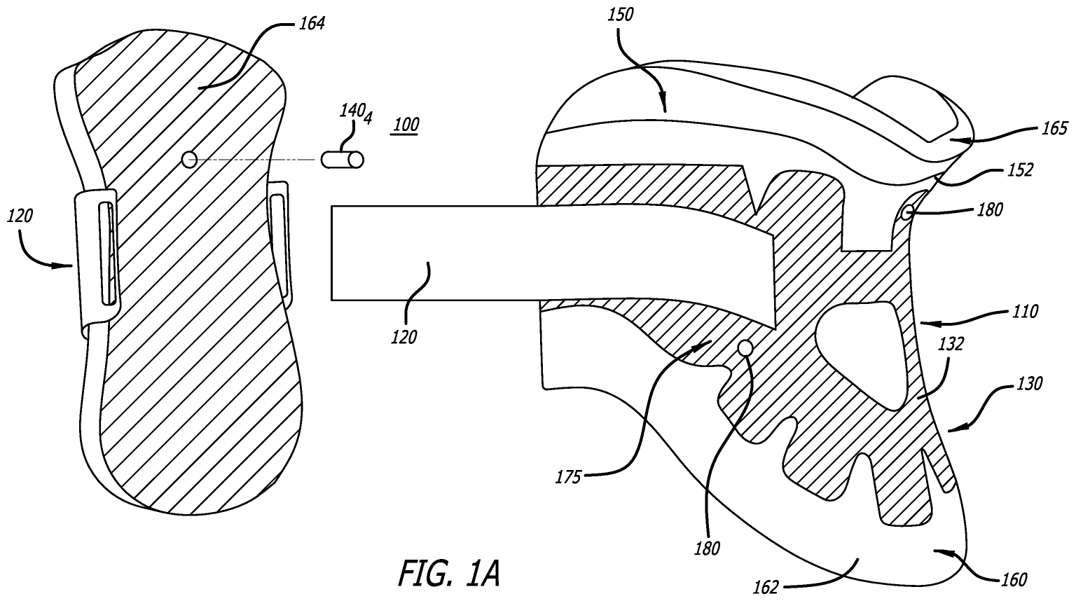

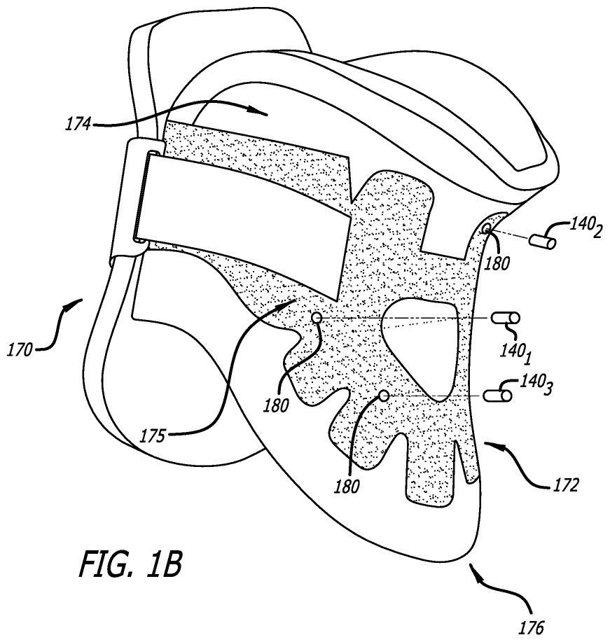

Referring to FIG. 1A, a perspective view of an exemplary embodiment of an orthopedic precast 100 is shown, where the orthopedic precast 100 is sized and dimensioned to wrap about a neck of a patient and operate (after customization) as a cervical orthosis. The orthopedic precast 100 may be configured as a front precast 110 and/or a back precast 120. The back precast 120 may be deployed as a separate element to be attached to the front precast 110 by straps and/or fasteners. Alternatively, although not shown, the back precast 120 may be deployed as a swing arm (e.g., attached to a rear lateral side of the front precast 110 and pivotable to attached to the other rear lateral side of the front precast 110) resulting in the cervical orthosis 170 as shown in FIG. 1B.

As shown in FIG. 1A, a first portion 130 of the front precast 110 may be interwoven with one or more strands of a first thermoplastic material 132, which transforms the first portion 130 of the front precast 110 into a rigid construction after heat is applied to the first portion 130 that exceeds a melting temperature of the first thermoplastic material 132. The interweaved pattern 175 of the strands of the first thermoplastic material 132 (and/or other strands of material forming the first portion 130) is configured to generate recessed areas 180 within the resultant first cervical orthosis 170 of FIG. 1B. The recessed areas 180 are sized to receive one or more electronic components such as sensors 140₁-140_{N} (N≥1). The sensors 140₁-140_{N} may include, but are not limited or restricted to one or more of the following: (i) an accelerometer 140₁ to detect a change in three-dimensional positioning and/or gravitational acceleration so as to measure tilt, vibration (e.g., leg weakness, etc.), of course, a fall; (ii) a moisture sensor 140₂ to detect a lack of dryness on a wound or surgical incision area for the patient; (iii) a motion sensor 140₃ to detect usage of the orthosis; and/or (iv) a temperature sensor 140₄ to detect a temperature of the patient to monitor for inflection. It is contemplated that other electronic components, such as wireless transmitters or wireless transceivers for example, may be coupled to the sensors 140₁-140_{N} and housed within one of the sensor housings.

Featured above and below the first portion 130 of the front precast 110, an upper portion 150 and a lower portion 160 of the front precast 110 may be interwoven with strands of non-thermoplastic material 152 and 162 to retain their elasticity and flexibility after heat exceeds the melting temperature of the thermoplastic material 132 is applied. Additionally, or in the alternative, the upper and lower portions 150 and 160 may be interwoven with strands of a second (or third) thermoplastic material, provided the second (or third) thermoplastic material is different than the first thermoplastic material 132 and has a melting temperature that greatly exceeds the melting temperature of the first thermoplastic material 132. One or more sensors 140₁-140_{N} may be retained within the upper and lower portions 150 and 160, most notably the accelerometer 140₁ to monitor for unwanted tilting of the neck and the moisture sensor 140₂ positioned in a chin rest area 165 of the front precast 110.

According to one embodiment of the disclosure, as shown in FIGS. 1A-1B, the back precast 120 may include one or more strands of a thermoplastic material 164 so that, after applying heat at or exceeding its melting temperature, the thermoplastic material 164 may diffuse to form a curved orientation to operate as a head rest. The back precast 120 may include one or more recessed areas 190 to receive one or more of the sensors 140₁ . . . or 140_{N} (e.g., sensor 140₄). The front precast 110 and the back precast 120 generally compose a tube 195 when the cervical orthosis 170 is placed into a closed (worn) position.

Herein, according to one embodiment of the disclosure, each strand of the one or more strands forming the first interwoven portion 130 may include at least one type of thermoplastic material. For example, the strand(s) forming the first interwoven portion 130 may include a single type of thermoplastic material. Alternatively, the strand(s) forming the first interwoven portion 130 may constitute a composite, which may include at least two different thermoplastic materials, where the thermoplastic materials may have the same or different melting temperatures. As yet another alternative embodiment of the disclosure, the strand(s) can also be a composite of one or more thermoplastic materials and one or more non-thermoplastic materials, provided the rigidity of the first interwoven portion 130 is altered after a selected temperature and duration of heat is applied and the first interwoven portion 130 is cooled. The strand(s) may be formed from the thermoplastic material(s) or the strand(s) of a different material may be coated and/or impregnated (through an application process) with one or more thermoplastic materials. Herein, “thermoplastic material” may reference a collection of material inclusive of one or more strands of thermoplastic material that, when heated and cooled, alter the overall rigidity of the material while “non-thermoplastic material” is devoid of thermoplastic material to affect such rigidity.

According to one embodiment of the disclosure, the thermoplastic materials may be configured to form flexible strands at room temperature, are non-toxic, melt between 140° C. and 350° C., and become generally rigid when strands are partially melted together into a sheet or mat having a prescribed thickness, which may range from 0.5 millimters (mm) to 6 mm for example. Contemplated examples of thermoplastic materials may include, but are not limited or restricted to the following: Polyethylene Terephthalate (PET), Polyether ether ketone (PEEK), Polyphenylene oxide (PPO), Polypropylene (PP), polyethylene (PE), polyvinyl chloride (PVC) and polystyrene (PS), poly(methyl methacrylate) (PMMA), Acrylonitrile butadiene styrene (ABS), Polylactic acid (PLA), Polybenzimidazole (PBI), Polycarbonate (PC), Polyether sulfone (PES), Polyoxymethylene (POM), Polyphenylene sulfide (PPS), Polystyrene, Polyvinyl chloride (PVC), Polyvinylidene fluoride (PVDF), Polytetrafluoroethylene (PTFE), Polyamide 6 (PA6), Polybutylene terephthalate (PBT), Polyetherimide (PEI), or the like.

The thermoplastic and non-thermoplastic materials can be selected using any combination of natural and synthetic materials to accomplish a desired characteristic, as for example, a desired degree of stiffness, compressibility, flexibility, bending, stretch, and resilience. As described above, one or more strands associated with the first interwoven portion 130 of the orthopedic precast 100 may include both thermoplastic and non-thermoplastic materials. For example, the non-thermoplastic material may include Kevlar™ to increase the durability/toughness of the resulting orthosis 170, as one or more other non-thermoplastic materials (e.g., cotton fibers, non-carbon fibers, nanotubes, glass fibers, ceramic, and/or metal fibers) may be used. Similarly, one or more strands of material forming the upper/lower portions 150 and/or 160 can also comprise thermoplastic and/or non-thermoplastic materials. However, according to this embodiment of the disclosure, the thermoplastic material of the upper/lower portions 150 and/or 160 may feature a higher melting temperature that is substantially above the melting temperature of the thermoplastic material used in the first portion 130.

As a result, as shown in FIG. 1B, one of the inventive concepts is that a precast forming the cervical orthosis 170 will have (1) one or more strands of a first (thermoplastic) material interwoven with similar strands of a different material, which upon heating, partially melt and therefore fuse together to form the rigid shell portion 172, and (2) one or more interwoven strands of a second (non-thermoplastic) material that remain flexible upon cooling, either because they do not melt, or they melt to an insubstantial amount at the temperature used to melt the thermoplastic materials forming the shell portion 172, collectively form the upper/lower portions 174/176 of the resulting cervical orthosis 170. Accordingly, the terms “insubstantial” and “substantial” are used herein in that context as to the amount of thermoplastic material melting to change a portion of the precast of the orthosis into a rigid state.

It should be appreciated then, that the one or more interwoven strands of a different material or materials that remain flexible upon cooling might or might not include a thermoplastic material. Preferably, however, the one or more interwoven strands of a different material or materials that remain flexible upon cooling could mostly or entirely comprise a natural fiber such as cotton or wool. To avoid oxidation of such non-thermoplastic materials, heating can take place in an anoxic or low oxygen environment.

In production, as shown in FIGS. 1A-1B, the orthopedic precast 100 may be placed over a positive mold and heated, such that at least some of the thermoplastic material(s) fuse, in what will become a rigid shell. This allows the resultant shell portion 172 (produced from the first interwoven portion 130) to closely conform to whatever part(s) of the patient are to be motion-restrained. Alternatively, the orthopedic precast 100 may be placed over a first positive mold, removed inside-out to provide a different (opposite) layering scheme for this flipped precast, which is placed over a second positive mold, heated, and subsequently cooled to form the cervical orthosis 170 with the rigid shell portion 172 or the cervical orthosis 170 with multiple layers.

As shown, the orthopedic precast 100 may feature a tubular configuration, with superior and inferior open ends. However, as depicted in FIG. 1B, the resultant cervical orthosis 170 can also open and close laterally, using a fastener such as a Velcro™ or similar hook and loop fastener, which can be installed at the precast stage. Any suitable fasteners are contemplated, including buttons, toggles, studs, snap fastener, poppers, buckles, zippers, frogging, hooks and eyes, magnets, grommets, brooches, safety pins, fabric ties, and laces.

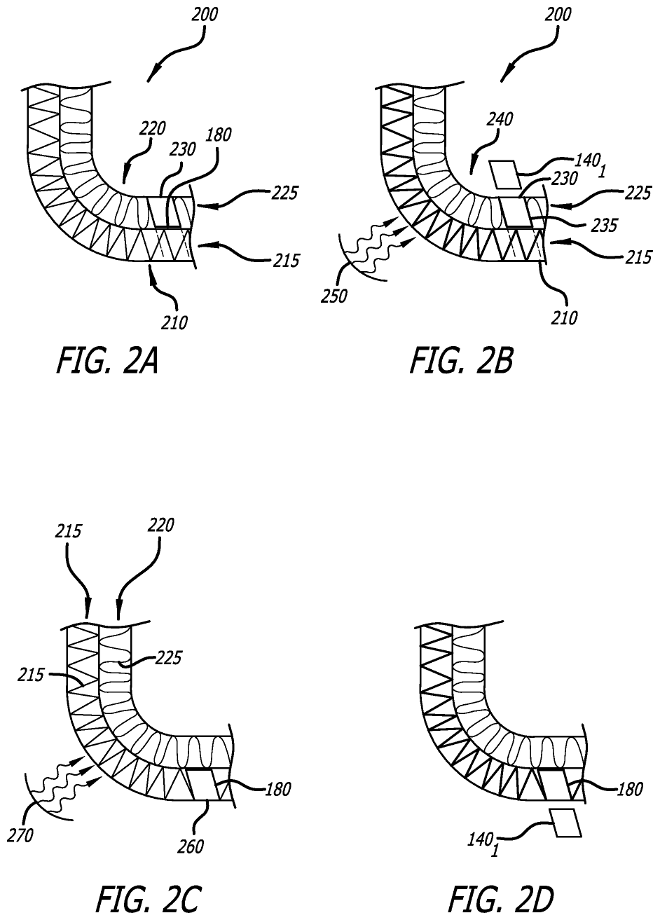

Referring now to FIG. 2A, a first exemplary embodiment of a portion of the orthopedic precast 100 of FIG. 1A with strands of thermoplastic material formed as a layer of the cervical orthosis 170 is shown. This portion 200 of the orthopedic precast includes a first interwoven portion 210 and a second interwoven portion 220. Herein, the first interwoven portion 210 features one or more strands of a first material 215, namely a first thermoplastic material having a first melting temperature (T1). The second interwoven portion 220 features one or more strands of a second material 225, namely a non-thermoplastic material or a second thermoplastic material having a second melting temperature (T2) that substantially exceeds the first melting temperature (T2>>T1). For this illustrative embodiment, in some regions, the different materials 215 and 225 may be layered and arranged with an interweaved pattern that includes at least one opening 230 within the second material 225. The opening 230 may extend from an internal surface 240 of the orthopedic precast portion 200 through the second material 225 of the second interwoven portion 220 and into or through the first material 215 of the first interwoven portion 210. The opening 230 may provide the recessed area 180 with exposure to a rigid chamber 235 (see FIG. 2B) formed by the strands of the first material 215 without interference by the strands of the second material 225.

According to one embodiment of the disclosure, the heat may be provided by a remote heat source 250 that produces a heated ambient environment, produces heated air flow, or produces heated steam. Additionally, or in the alternative, the heat may be provided by an interconnect (not shown) operating as a heating element integrated proximate to the strands of the first thermoplastic material exposed by the opening 230.

Based on this interweaved pattern, upon applying heat to the portion of the first interwoven portion 210 that exceeds the first melting temperature (T1) and below the second melting temperature (T2), the first material 215 partially melts and diffuses to form recessed area 180 with the opening 230 providing access to the recessed area 180. Upon ceasing the application of heat and allowing the portion 200 of the orthopedic precast 100 to cool, a hardened recessed area 180 is created as shown in FIG. 2B. The size of the opening enables the recessed area 180 to receive and securely retain a sensor (e.g., sensor 140₁) of the sensors 140₁-140_{N}. In general terms, after cooling, the first material 215 solidifies and becomes rigid to form the rigid recessed area 180 while the second material 225 retains the same phase and operates as a soft interface between the rigid, first interwoven portion 210 and a skin of the patient.

Referring now to FIG. 2C, an exemplary embodiment of the portion 200 of the orthopedic precast 100 in FIG. 2A is shown, following heating at or above the lower range of the melting temperature of the first thermoplastic material 215 of the first interwoven portion 210. This portion of the orthopedic precast 100 includes the first interwoven portion 210 and the second interwoven portion 220. Herein, the first interwoven portion 210 features one or more strands of the first thermoplastic material 215 having the first melting temperature (T1) while the second interwoven portion 220 features one or more strands of non-thermoplastic material and/or the second thermoplastic material having the second melting temperature (T2). For this illustrative embodiment, in some regions, the strands of the first thermoplastic material 215 may be arranged with one or more exterior facing openings 260 for the sensors 140₁-140_{N}. The openings 260 provide exposure directly to the strands of the first material 215 without interference by the strands of the second material 225.

According to one embodiment of the disclosure, the heat may be provided by a remote heat source 270 that produces a heated ambient environment, produces heated air flow, or produces heated steam. Based on this interweaved pattern, upon applying heat from the heat source 270 that exceeds the first melting temperature (T1) and remains below the second melting temperature (T2), the first material 215 partially melts and diffuses to form recessed area 180 by its coating of the exterior facing opening 260.

As shown in FIG. 2D, upon ceasing the application of heat and allowing the precast 100 to cool, a hardened recessed area 180 is created. The size of the recessed area 180 (e.g., depth, width, or diameter, etc.) is intended to house a sensor (e.g., sensor 140₁) of the sensors 140₁-140_{N}. In general terms, after cooling, the first material 215 solidifies and becomes rigid to form the hardened recessed area 280 to house any of the sensors 140₁-140_{N} described above.

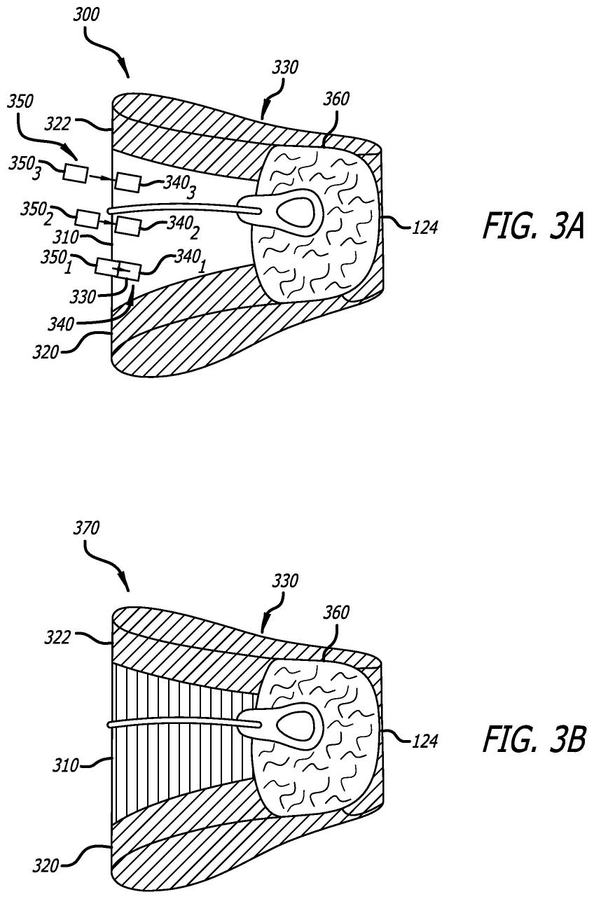

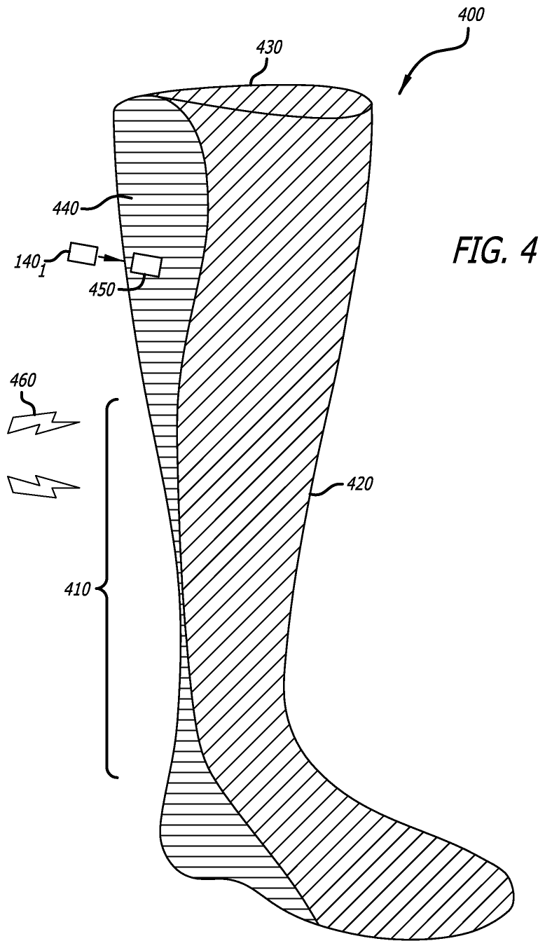

Referring to FIG. 3A, an exemplary embodiment of an orthopedic precast 300 from which an orthosis, sized and dimensioned to wrap about a torso of a patient and operate as a lumbar sacral orthosis (LSO) or part of a thoracic lumbar sacral orthosis (TLSO), is shown. The orthopedic precast 300 may be configured with a first portion 310 interwoven with one or more strands comprising thermoplastic material, which features a rigid construction after a prescribed amount of heat is applied to the first portion 310 (referred to as a “first interwoven portion”). Featured above and below the first interwoven portion 310, upper and lower portions 320 and 322 may be attached with one or more strands comprising non-thermoplastic material (each referred to as a “flexible portion”). As shown, for this embodiment of the disclosure, the first interwoven portion 310 and the flexible portions 320, 322 generally compose a tube (e.g., belt) 330, with two open ends.

According to one embodiment of the disclosure, the interweaving of the first interwoven portion 310 and/or the flexible portions 320, 322 may be oriented to create one or more recessed areas 340 with to retain one or more (M) sensors (hereinafter, “sensor”) 350 of the belt 330. As described above, the sensor(s) 350₁-350_{M} may include, but are not limited or restricted to an accelerometer 350₁ placed within recessed area 340₁ to detect tilt or a fall; a moisture sensor 350₂ placed within recessed area 340₂ to detect a lack of dryness on a wound or surgical incision area for the patient, and/or a motion sensor 350₃ placed within recessed area 340₃ to detect usage of a resultant orthosis 370 as shown in FIG. 3B.

According to another embodiment of the disclosure, the interweaving of the first interwoven portion 310 may be oriented inferior-superior (e.g., vertically) to allow for anterior/posterior movement of the belt 330 despite the first interwoven portion 310 being rigid in construction. The orientation of the interweaving of the thermoplastic material (by itself or in combination with non-thermoplastic material) permits lateral movement of arms 360 of the resultant orthosis 370.

Referring to FIG. 4, an exemplary embodiment of an orthopedic precast 400, which can be heated and cooled to produce an ankle-foot-orthosis (AFO) configured to restrict movement of lower leg of a patient relative to the foot, is shown. The precast 400 generally includes a first interwoven portion 410 and a second interwoven portion 420 generally configured as a long sock featuring a tube 430 having an open upper calf end and a closed toe end. The second interwoven portion 420 may be formed with elastic, non-thermoplastic material operating as a flexible portion for the precast 400 (as well as the resultant orthosis) while the first interwoven portion 410 may be formed with thermoplastic material operating as the shell portion for the precast (and resultant orthosis).

As shown, the first interwoven portion 410 may feature strands of thermoplastic material 440 having a melting temperature (T1), which is interweaved in accordance with a prescribed pattern to create one or more recessed areas 450 for sensor deployment in response to the strands of thermoplastic material 440 undergoing a phase change. More specifically, upon applying heat 460 at or exceeding a targeted temperature (T1), the thermoplastic material associated with the strands of thermoplastic material 440 melts and diffuses to form the recessed area(s) 450 after the melted thermoplastic material 440 cools.

The recessed area(s) 450 are sized to retain any of a number of sensors, including an accelerometer 140₁ to monitor for sufficient support and proper swing as shown. Other sensors may include a moisture sensor, a motion sensor to monitor usage, and/or a temperature sensor to monitor for patient health and potential infections when the resultant AFO will monitor for post-operative inflection.

The precast 400 could also be provided in a preformed condition, in an average shape of a given anatomical size. Various sizes could be offered accordingly. This pre-shaped item could be commercially offered as an “off-the-shelf” product that could be provisioned to a patient of average contours given their dimensions without modification. It could also provide the opportunity for optimization of the contours through heating and reforming the shell material(s) in strategic locations.

In a corresponding precast 400, the first interwoven portion 410 and the second interwoven portion 420 cooperate to support posterior and anterior parts of the lower leg, respectively. The first interwoven portion 410 and the second interwoven portion 420 are oriented lengthwise along the tube 430. In this configuration, after conversion of the precast into an orthosis, the second interwoven (flexible) portion 420 allows for easier donning and removal of the orthosis, while the first interwoven (shell) portion 410 provides dorsiflexion, plantarflexion, inversion, and eversion stability at the ankle.

Strategic use of elastic regions can enhance functionality. For example, in FIG. 4, the second interwoven (flexible) portion 420 can be elastic, and such elasticity can function to press the first interwoven (shell) portion 410 against the back of the leg, and that can assist in lifting the foot during swing phase of ambulation. The accelerometer 140₁ located in the recessed area 450, can assist in monitoring of suitable swing phase where rigidity of the resultant shell portion 410 may assist in ensuring that there is no bias caused by motion within an elastic material. In some embodiments, an orthosis derived from the precast 400 can have a slightly dorsiflexed configuration such that when worn, the weight of the foot pulls the foot into a neutral (neither dorsiflexed nor plantarflexed) or other desired configuration.

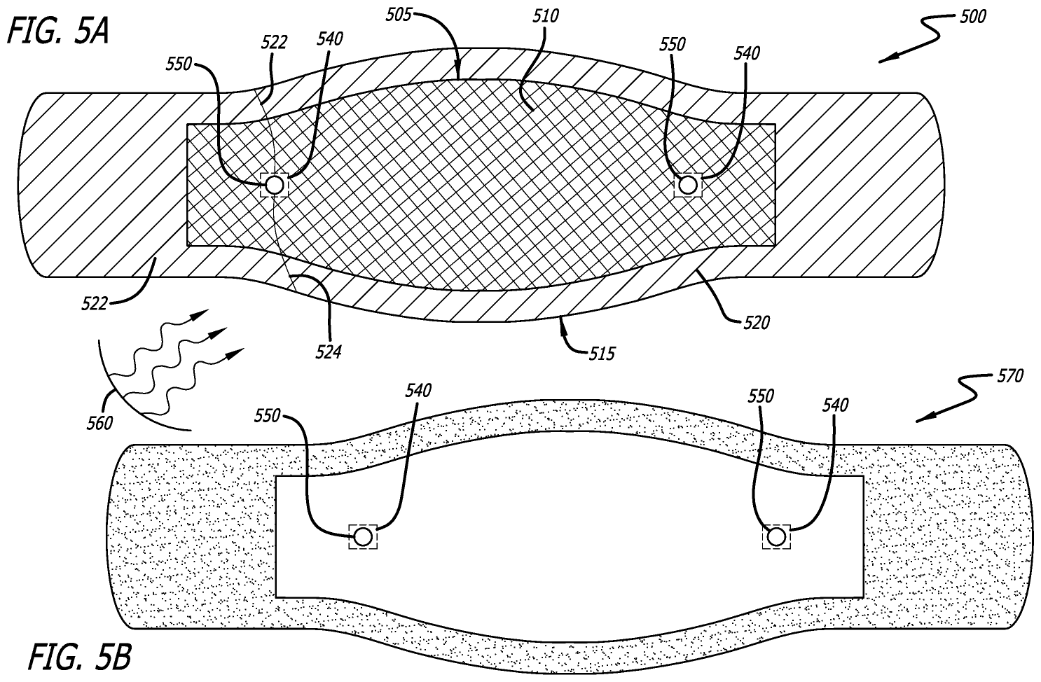

Referring now to FIG. 5A, a second exemplary embodiment of a portion of an orthopedic precast 500 with interwoven strands of thermoplastic material 510 to operate as region of a resultant orthosis 570 (see FIG. 5B) is shown. The orthopedic precast 500 includes the strands of a first thermoplastic material 510 and/or strands of a second material 520 arranged in a grid configuration to create one or more recessed areas 540 to partially house one or more sensing elements 550 (e.g., sensors, control units, etc.) positioned within the orthopedic precast 500.

More specifically, according to one embodiment of the disclosure, the orthopedic precast 500 features a first interwoven portion 505 including strands of the first thermoplastic material 510 having a first melting temperature (T1). As an optional feature, the orthopedic precast 500 may include a second interwoven portion 515 that features strands of one or more other materials 520, such as a non-thermoplastic material 522 and/or a second thermoplastic material 524 having a second melting temperature (T2) that substantially exceeds the first melting temperature (T2>>T1). For example, the strands of the second thermoplastic material 524 may feature conductive interconnects, which are communicatively coupled to the sensing element(s) 550 and operate as an ancillary element to collect data for the sensing element(s) 550.

For this illustrative embodiment, in some regions, the first interwoven portion 505 may be configured with sensing elements 550 positioned into the recessed areas 540 before or after activation of the first thermoplastic material 510. The activation of the first thermoplastic material 510 may be accomplished by applying heat 560 that exceeds T1, which causes the first thermoplastic material 510 to experience a phase change from a flexible (fabric-like) state to a rigid state as shown in FIG. 5B. The sensing elements 550 may be re-positioned after the phase change (and application of prescribed heat) or may be positioned before the application of heat. One of the sensing elements 550 may include an accelerometer to detect a change in gravitational acceleration, making it possible to measure tilt, vibration that signify weakness of a body part of a patient, or a fall represented by a significant and immediate change. Another of the sensing elements 550 may include a motion sensor to detect usage of the orthosis where prolonged non-movement may signify non-use of the orthosis.

More specifically, based on this interweaved pattern, upon applying heat to the portion of the first interwoven portion 505 that exceeds the first melting temperature (T1) and below the second melting temperature (T2), the first thermoplastic material 510 partially melts and diffuses to define a recessed area 540. Depending on the grid layout, the sensing element 550 may have access the skin of a patient. Upon ceasing the application of heat and allowing the first interwoven portion 505 of the orthopedic precast 500 to cool, one or more hardened recessed area 540 are created as shown in FIG. 5B. The size of the recessed areas 540 enables are adapted to receive and securely retain a sensor from the sensing elements 550. In general terms, after cooling, the first thermoplastic material 510 solidifies and becomes rigid to form the rigid recessed area 540 while the non-thermoplastic material 522 retains the same phase.

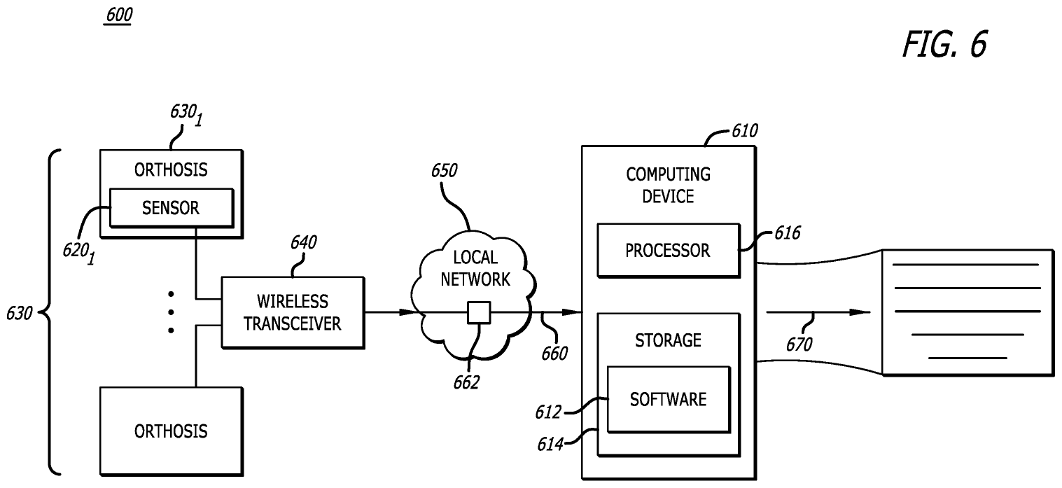

Referring now to FIG. 6, an exemplary embodiment of a system 600 featuring a computing device 610 is shown, which is adapted for communications with sensors 620 associated with one or more orthoses 630 and configured to conduct analytics of data captured by the sensors 620. Herein, according to this illustrative embodiment, sensor 620₁ deployed within a first orthosis 630₁ may be communicatively coupled to a wireless transceiver 640 co-located within first orthosis 630₁. The wireless transceiver 640 may operate as a low-range transceiver, such as a Bluetooth™ transceiver or example, to communication with a local network 650 to upload data monitored or measured by the sensor 620₁.

For example, where the sensor 620₁ is an accelerometer located within the first orthosis 630₁ operating as a cervical orthosis for example, the sensor 620₁ may measure a degree of neck tilt being experienced by the cervical orthosis 630₁ when worn by the patient or falling by the patient. Measured tilt parameters 662 are provided, via the wireless transceiver 640 and local network, as part of signaling 660 from the sensor 620₁ to the computing device 610 via the wireless transceiver 640. The computing device 610 may include software 612, stored within non-transitory storage medium 614 and processed by a processor 616, that determines whether any of the measured tilt parameters exceed a prescribed tilt threshold. If so, a doctor or an orthotist may be notified from the computing device via a second signaling 670 for orthosis adjustment (e.g., text, email message, etc.) for display of the reported measured tilt data. With respect to a fall, the tilt parameters may exceed a thresholds (e.g., amount of tilt and rate of change associated with the tilt). The second signaling 670 may provide a higher-level alert to caregivers and/or emergency response providers.

According to another example, located within the cervical orthosis 630₁, the sensor 620₁ may operate as motion sensor to detect usage of the cervical orthosis 630₁. Based on parameters received by the wireless transceiver 640 that denote motion undertaken by the motion sensor 620₁ or denote the orientation of the motion sensor 620₁, the wireless transceiver 640 and local network 650 are configured to provide signaling 660 inclusive of the detected motion and/or orientation. The software 612 installed within the computing device 610 is configured to determine whether the patient is complying with instructed usage of the cervical orthosis 630₁. If not, the patient may receive a warning message of non-compliant usage and/or the doctor/orthotist may be notified of the same.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refer to at least one of something selected from the group consisting of A, B, C . . . and N, the text should be interpreted as requiring only one element from the group, not A plus N, or B plus N, etc.

What is claimed is:

1. An orthopedic precast for use in combinations of the orthopedic, comprising:
a first portion including a first plurality of interwoven strands of a first thermoplastic material, the first thermoplastic material having a first melting temperature;
a second portion coupled to the first plurality of interwoven strands of the first thermoplastic material; and
one or more sensors, wherein
responsive to applying heat of at least the first melting temperature to the first plurality of interwoven strands of the first thermoplastic material and subsequent cooling, the first plurality of interwoven strands of the first thermoplastic material are interweaved to create one or more recessed area for receipt of the one or more sensors.

2. The orthopedic precast of claim 1 transitions to an orthosis upon the first portion transitioning from a flexible state to a rigid state after partial melting and subsequent cooling of the first plurality of interwoven strands of the first thermoplastic material.

3. The orthopedic precast of claim 2, wherein the one or more sensors include an accelerometer to detect a change in gravitational acceleration to measure a tilt of the orthosis or a fall of a patient wearing the orthosis.

4. The orthopedic precast of claim 2, wherein the one or more sensors include a moisture sensor to detect a lack of dryness on a wound area proximate placement of the orthosis when worn by a patient.

5. The orthopedic precast of claim 2, wherein the one or more sensors include a motion sensor to detect usage of the orthosis.

6. The orthopedic precast of claim 2, wherein the one or more sensors include a temperature sensor to detect a potential infection associated with a wound or a surgical incision proximate to placement of the orthosis when worn by a patient.

7. An orthopedic precast for use in combinations of the orthopedic, comprising:
one or more layers of interwoven strands of a first thermoplastic material, the first thermoplastic material having a first melting temperature; and
one or more sensors, wherein
responsive to applying heat of at least the first melting temperature to the one or more layers of the interwoven strands of the first thermoplastic material and subsequent cooling, the one or more layers of interwoven strands of the first thermoplastic material are interweaved to create one or more recessed area within an orthosis formed in part by the one or more layers of the interwoven strands of the first thermoplastic material after heated for receipt of the one or more sensors.

8. The orthopedic precast of claim 7, wherein the one or more sensors are positioned within the one or more layers of interwoven strands of the first thermoplastic material prior to the applying of the heat to the one or more layers.

9. The orthopedic precast of claim 7 is configured to transition to an orthosis upon the one or more layers of the interwoven strands of the first thermoplastic material transitioning from a flexible state to a rigid state after partial melting and subsequent cooling of the one or more layers of the interwoven strands of the first thermoplastic material.

10. The orthopedic precast of claim 7, wherein the one or more sensors include an accelerometer to detect a change in gravitational acceleration to measure a tilt of the orthosis or a fall of a patient wearing the orthosis.

11. The orthopedic precast of claim 7, wherein the one or more sensors include a moisture sensor to detect a lack of dryness on a wound area proximate placement of the orthosis when worn by a patient.

12. The orthopedic precast of claim 7, wherein the one or more sensors include a motion sensor to detect usage of the orthosis.

13. The orthopedic precast of claim 7, wherein the one or more sensors include a temperature sensor to detect a potential infection of a wound or a surgical incision proximate to placement of the orthosis when worn by a patient.

14. An orthosis formed from an orthopedic precast, comprising:
a first portion formed from one or more layers of interwoven strands of a first thermoplastic material after an application of a prescribed amount of heat and subsequently cooled; and
one or more sensors positioned within the first portion of the orthosis.

15. The orthosis of claim 14, wherein the first portion corresponds to a front portion of a cervical collar.

16. The orthosis of claim 14, wherein the one or more sensors include an accelerometer to detect a change in gravitational acceleration to measure a tilt of the orthosis or a fall of a patient wearing the orthosis.

17. The orthosis of claim 14, wherein the one or more sensors include a moisture sensor to detect a lack of dryness on a wound area proximate placement of the orthosis when worn by a patient.

18. The orthosis of claim 14, wherein the one or more sensors include a motion sensor to detect usage of the orthosis.

19. The orthosis of claim 14, wherein the one or more sensors include a temperature sensor to detect a potential infection of a wound or a surgical incision proximate to placement of the orthosis when worn by a patient.
